(19) United States
(12) Patent Application Publication
(10) Pub. No.: US 2020/0155831 A1
(43) Pub. Date: May 21, 2020

(54) NASAL NEUROSTIMULATOR WITH INTEGRATED RFID

(71) Applicant: ALLERGAN / Oculeve, Madison, NJ (US)

(72) Inventors: John Wardle, San Clemente, CA (US); Jarren A. Baldwin, Oakland, CA (US); Nithin Dhananjayan, Fair Oaks, CA (US)

(21) Appl. No.: 16/685,984

(22) Filed: Nov. 15, 2019

Related U.S. Application Data
(60) Provisional application No. 62/768,685, filed on Nov. 16, 2018.

(51) Int. Cl. A61N 1/05; A61N 1/36; G06K 19/07

(52) U.S. Cl. CPC A61N 1/0546; A61N 1/0456; G06K 19/0723; A61N 1/36046

(57) ABSTRACT

Various embodiments of a handheld stimulator system is described that is configured to provide a stimulation to nasal tissue of a subject. In some embodiments, the handheld stimulator system includes a stimulator probe and a stimulator body, and the stimulator probe may be configured to releasably couple to the stimulator body. The stimulator probe may include a first nasal insertion prong comprising a first electrode and a tag of an RFID control mechanism that is configured to control a use of the stimulator probe. In some embodiments, the stimulator body may include a power source and a reader of the RFID control mechanism. The reader may be configured to communicate with the tag for assisting with controlling the use of the stimulator probe. Systems and method associated with the handheld stimulator system are also described.

### CROSS-REFERENCE TO RELATED APPLICATION

The current application claims priority under 35 U.S.C. § 119(e) to U.S. Provisional patent application Ser. No. 62/768,685, filed on Nov. 16, 2018 and entitled “Nasal Neurostimulator with Integrated RFID,” which is incorporated by reference herein in its entirety.

### TECHNICAL FIELD

The subject matter described herein relates to a handheld nasal stimulator and related methods of use.

### BACKGROUND

Dry Eye Disease (“DED”) is a condition that affects millions of people worldwide. More than 40 million people in North America have some form of dry eye, and many millions more suffer worldwide. DED results from the disruption of the natural tear film on the surface of the eye, and can result in ocular discomfort, visual disturbance and a reduction in vision-related quality of life. Activities of daily living such as driving, computer use, housework and reading have also been shown to be negatively impacted by DED. Patients with severe cases of DED are at risk for serious ocular health deficiencies such as corneal ulceration, and can experience a quality of life deficiency comparable to that of moderate-severe angina.

The etiology of DED is becoming increasingly well understood. DED is progressive in nature, and fundamentally results from insufficient tear coverage on the surface of the eye. This poor tear coverage prevents healthy gas exchange and nutrient transport for the ocular surface, promotes cellular desiccation and creates a poor refractive surface for vision. Poor tear coverage typically results from: 1) insufficient aqueous tear production from the lacrimal glands (e.g. secondary to post-menopausal hormonal deficiency, auto-immune disease, LASIK surgery, etc.), and/or 2) excessive evaporation of aqueous tear resulting from dysfunction of the meibomian glands. Low tear volume causes a hyperosmolar environment that induces an inflamed state of the ocular surface. This inflammatory response induces apoptosis of the surface cells which in turn prevents proper distribution of the tear film on the ocular surface so that any given tear volume is rendered less effective. This initiates a vicious cycle where more inflammation can ensue causing more surface cell damage, etc. Additionally, the neural control loop, which controls reflex tear activation, is disrupted because the sensory neurons in the surface of the eye are damaged. As a result, fewer tears are secreted and a second vicious cycle develops that results in further progression of the disease (fewer tears cause nerve cell loss, which results in fewer tears, etc.). Accordingly, effective treatment for dry eye is desired.

### SUMMARY

Aspects of the current subject matter include various embodiments of a handheld stimulator system configured to provide a stimulation to a subject. The handheld stimulator system may include a nasal stimulator probe and a stimulator body. In one aspect consistent with the current disclosure, a handheld stimulator system for stimulating nasal tissue of a subject may include a stimulator probe. The stimulator probe may include a first nasal insertion prong. The first nasal insertion prong may include a first electrode. The stimulator probe may include a tag of an RFID control mechanism. The RFID control mechanism may be configured to control a use of the stimulator probe. The handheld stimulator system may include a stimulator body configured to releasably couple the stimulator probe thereto. The stimulator body may include a power source. The stimulator body may include a reader of the RFID control mechanism. The reader may be configured to communicate with the tag for assisting with controlling the use of the stimulator probe.

In some variations one or more of the following features can optionally be included in any feasible combination. In some embodiments, the tag may include a tag identifier that is read by the reader for authenticating the stimulator probe. The tag may be embedded within a material comprising the stimulator probe. The tag may be positioned adjacent a base of the stimulator probe. The base may couple with a coupling end of the stimulator body. The reader may be positioned along the coupling end of the stimulator body thereby allowing the reader to communicate with the tag when the stimulator probe is coupled to the stimulator body. The reader may be configured to communicate with the tag when the tag is positioned within a predefined distance of the reader. The tag may be positioned at the predefined distance when the stimulator probe is coupled to the stimulator body. The tag may be outside of the predefined distance when the stimulator probe is separated from the stimulator body.

An antennae of the reader may be positioned adjacent an end of an extension extending from the coupling end of the stimulator body. The extension may be configured to be inserted within a cavity along the base of the stimulator probe for positioning the antennae within the stimulator probe and adjacent the tag. The RFID control mechanism may include a programmed predefined threshold that controls a delivery of power from the power source to the first electrode. The RFID control mechanism may be configured to compare a received identifier associated with the tag against a list of stored acceptable tag identifiers and allow a current from the power source to be delivered to the first electrode if the received identifier matches an acceptable tag identifier of the list of stored acceptable tag identifiers. The RFID control mechanism may be configured to prevent current from the power source to the first electrode after a use parameter of the stimulator probe meets and/or exceeds the predefined threshold. The predefined threshold may define a duration of time or a date. The predefined threshold may define a number of times a stimulation is delivered. The RFID control mechanism may be configured to prevent current from the power source to the first electrode if an identifier associated with the tag fails to match an acceptable tag identifier in a list of acceptable tag identifiers stored on the stimulator body. The RFID control mechanism may be configured to include a use identifier on the tag after the stimulator probe is coupled to the stimulator body for assisting with limiting the use of the stimulator probe.

In another interrelated aspect of the current subject matter, a method includes coupling a stimulator probe of the handheld nasal stimulator to a stimulator body of the handheld nasal stimulator. The stimulator probe may include an electrode and a tag associated with an RFID control mechanism. The RFID control mechanism may be configured to control a use of the stimulator probe. The method of the handheld nasal stimulator may include detecting, after the coupling and by a reader of the RFID control mechanism, the tag of the stimulator probe. The stimulator body may include the reader configured to communicate with the tag when the tag is positioned within a predefined range from the reader. The method of the handheld nasal stimulator may include comparing, by the RFID control mechanism, an identifier associated with the tag against a list of acceptable identifiers stored on the stimulator probe. The method of the handheld nasal stimulator may include controlling, by the RFID control mechanism and based on the comparing, a delivery of current from a power source of the stimulator body to the electrode of the stimulator probe for delivering a stimulation.

In some embodiments, the controlling may include allowing the delivery of current from the power source to the electrode when the identifier associated with the tag matches an acceptable identifier of the list of acceptable identifiers. The controlling may include allowing the delivery of the current from the power source to the electrode when a use parameter of the stimulator probe meets and/or exceeds a programmed predefined threshold. The programmed predefined threshold may be a number of times the stimulation is delivered. The programmed predefined threshold may be a duration of time or a date. The controlling may include preventing the delivery of current from the power source to the electrode when the identifier associated with the tag fails to match an acceptable identifier of the list of acceptable identifiers. The method may include associating, by the reader and after the coupling, a use identifier with the tag for limiting a use of the stimulator probe.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIGS. 1A, 1B, 1C, 1D, 1E illustrate perspective, front, back, cut-away back, and cut-away side views, respectively, of an illustrative variation of a handheld stimulator;

FIG. 2 illustrates a block diagram schematically representing a variation of a stimulator;

FIG. 3A illustrates a perspective view of another embodiment of a handheld simulator including an RFID control mechanism and a stimulator probe configured to releasably couple to a stimulator body;

FIG. 3B illustrates a front perspective view of the stimulator body of FIG. 3A;

FIG. 3C illustrates the stimulator body of FIG. 3B with a top cover removed to show a part of a reader antenna of a reader component of the RFID control mechanism;

FIG. 3D illustrates the reader component of the RFID control mechanism positioned within the stimulator body of FIG. 3A;

FIG. 3E illustrates a tag of the RFID control mechanism coupled to the stimulator probe of FIG. 3A;

FIG. 3F illustrates a partial cross-section side view of the handheld stimulator of FIG. 3A taken along a centerline extending between nasal insertion prongs of the stimulator probe and with the stimulator probe coupled to the stimulator body, and the partial cross-section side view of FIG. 3F shows the position of the tag relative to the antenna when the stimulator probe is coupled to the stimulator body;

FIG. 4A illustrates a front view of another embodiment of a stimulator probe configured to releasably couple to a stimulator body;

FIG. 4B illustrates a side section view of the stimulator probe of FIG. 4A showing a tag embedded within the stimulator probe;

FIG. 4C illustrates a partial view of the stimulator probe of FIG. 4B showing the tag coupled to a platform of the stimulator probe;

FIG. 4D illustrates the platform of the stimulator probe of FIG. 4C showing a coupling or recessed feature configured to couple the tag thereto;

FIG. 5 illustrates an information flow diagram illustrating data that may be sent from a smartphone to the handheld stimulator of FIG. 3A; and

FIG. 6 illustrates a block diagram schematically representing a variation of the handheld stimulator including a communication and authorization schematic associated with an embodiment of an RFID control mechanism.

When practical, similar reference numbers denote similar structures, features, or elements.

### DETAILED DESCRIPTION

This disclosure describes devices, systems, and methods for treating one or more conditions (such as dry eye) by providing stimulation to nasal or sinus tissue. The devices and systems may be configured to stimulate nasal or sinus tissue. In some variations, the devices may comprise a stimulator body and a stimulator probe, where the stimulator probe comprises one or more nasal insertion prongs. The stimulus delivered by the stimulators described herein may be electrical. When the devices and systems are used to treat dry eye, the methods may comprise stimulating nasal or sinus tissue to increase tear production, reduce the symptoms of dry eye, or improve ocular appearance and/or health.

Some stimulator embodiments described herein may include one or more safety mechanisms. For example, some stimulators include a stimulator body that is configured to releasably couple a stimulator probe. Such stimulators described herein may include a safety mechanism configured to identify and authenticate a stimulator probe coupled to the stimulator body prior to allowing stimulation by the stimulator. Such a safety mechanism may prevent against use of counterfeit stimulator probes. Additionally or alternatively, some stimulator embodiments disclosed herein include a safety mechanism configured to limit a duration of use of a stimulator probe that is releasably coupled to a stimulator body. Such a safety mechanism may require a user to adhere to replacement requirements of the stimulator probe, which may assist with preventing or limiting biohazard exposure to the user. Various stimulators and safety mechanisms are described in greater detail below.

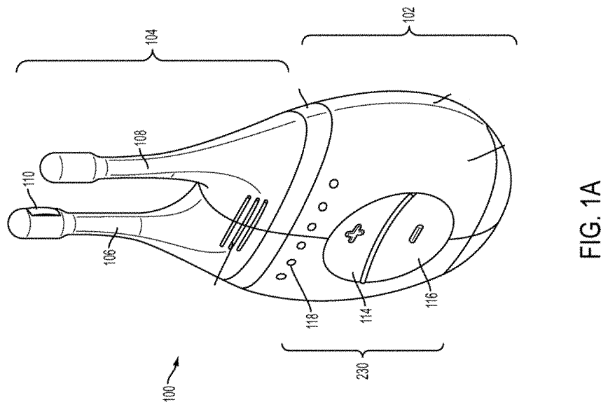

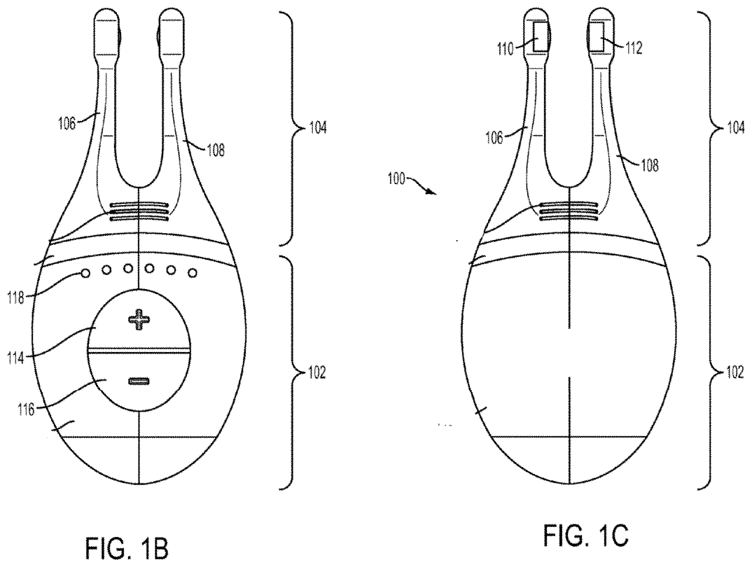

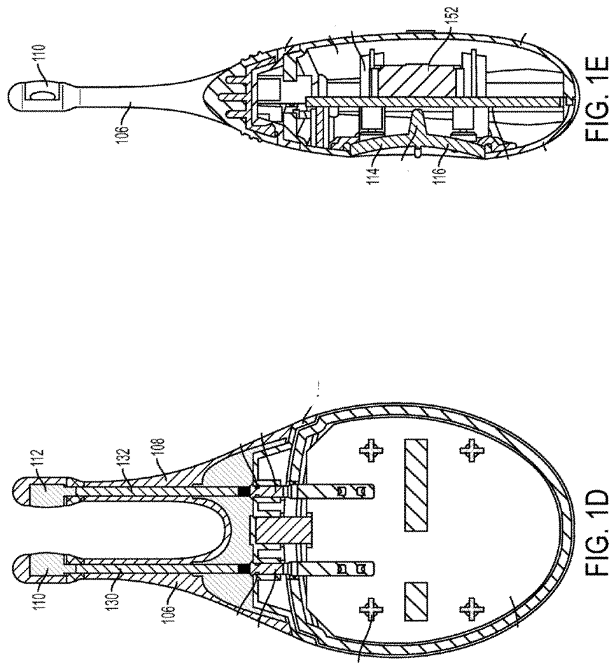

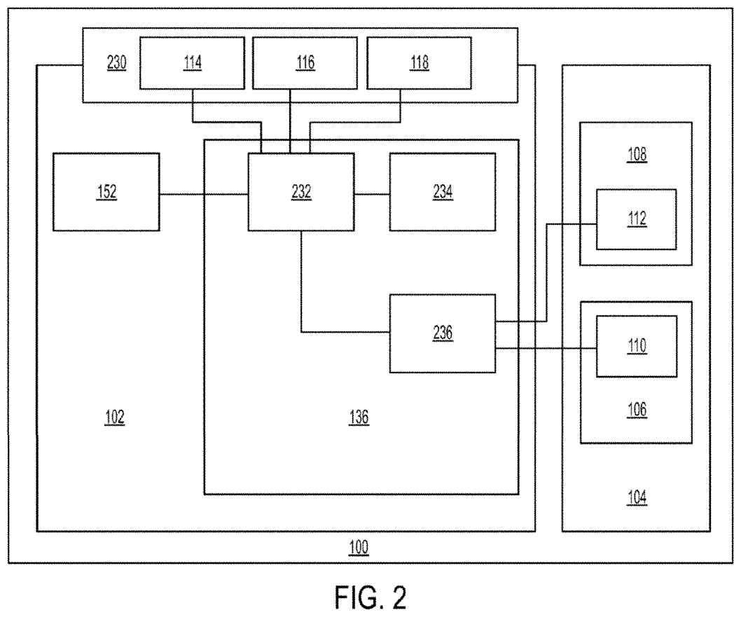

Some variations of the stimulation systems described herein may include a handheld stimulator. FIGS. 1A, 1B, 1C, 1D, 1E show perspective, front, back, cut-away back, and cut-away side views, respectively, of an illustrative variation of a handheld stimulator 100, respectively. FIG. 2 shows a block diagram schematically representing the stimulator 100. As shown in FIGS. 1A-1E, the stimulator 100 may comprise a stimulator body 102 and a stimulator probe 104. The stimulator body 102 may be configured to generate a stimulus that may be delivered to a subject. The stimulator body 102 may include a control subsystem 136 and a power source 152, which together may generate and control the stimulus.

The stimulus may be delivered to a subject via the stimulator probe 104. In some variations the stimulator body 102 and stimulator probe 104 may be reversibly attachable. Some or all of the stimulator 100 may be disposable. In other variations, one or more portions of the stimulator 100 may be reusable. For example, in variations where the stimulator probe 104 is releasably connected to the stimulator body 102, the stimulator body 102 may be reusable, and the stimulator probe 104 may be disposable and periodically replaced, as described in more detail below. The stimulator probe 104 may comprise at least one nasal insertion prong, which may be configured to be at least partially inserted into the nasal cavity of a subject or patient. In the handheld stimulator variation shown in FIGS. 1A-1E, the stimulator probe 104 may comprise two nasal insertion prongs 106 and 108.

In some variations, the stimulus may be electrical. In these instances, each nasal insertion prong 106, 108 may comprise at least one electrode. As shown, the probe 104 may comprise a first electrode 110 on nasal insertion prong 106 and a second electrode 112 on nasal insertion prong 108. As shown in the cut-away view of the stimulator 100 in FIG. 1D, the electrodes 110 and 112 may be connected to leads 130 and 132 located within prongs 106 and 108, respectively. The leads 130 and 132 may connect directly or indirectly to the control subsystem 136 and power source 152. As such, the electrical stimulus may travel from the control subsystem 136, through the leads 130 and 132, and through the electrodes 110 and 112.

The stimulator body 102 may comprise a user interface 230 comprising one or more operating mechanisms to adjust one or more parameters of the stimulus. The operating mechanisms may provide information to the control subsystem 136, which may comprise a processor 232, memory 234, and/or stimulation subsystem 236. In some variations, the operating mechanisms may comprise first and second buttons 114 and 116. In some variations, pressing the first button 114 may turn on the stimulator 100 and/or change one or more parameters of the stimulus (e.g., increase the intensity of the stimulus, change the stimulation pattern, or the like), while pressing the second button 116 may turn off the stimulator 100 and/or change one or more parameters of the stimulus (e.g., decrease the intensity of the stimulus, change the stimulation pattern, or the like). Additionally or alternatively, the user interface may comprise one or more feedback elements (e.g., based on light, sound, vibration, or the like). As shown, the user feedback elements may comprise light-based indicators 118, which may provide information to the user. Additionally or alternatively, in some variations the stimulator body may comprise a display, which may be configured to convey information to a user via text and/or images. Additionally or alternatively, the stimulator body may comprise a speaker or buzzer configured to produce one or more speech prompts or other sounds. Additionally or alternatively, the stimulator body may be configured to vibrate.

As discussed above, the stimulator 100 may comprise a power source 152. The power source may be any suitable power supply capable of powering one or more functions of the stimulator, such as one or more batteries, capacitors, or the like. While the stimulator body 102 comprises a power source, in other variations the stimulator body need not comprise a power source. In some variations, the stimulator body may comprise a port, cord, or other mechanism for connecting the stimulator to an external power source (such as a wall outlet or separate battery pack), which in turn may be used to power one or more portions of the stimulator.

Generally, the processor 232 may be configured to control operation of the various subsystems of the control subsystem 136. For example, the processor 232 may be configured to control the stimulation subsystem 236 to control parameters of the stimulation provided by the stimulation subsystem 236. The memory 234 may be configured to store programming instructions for the stimulator, and the processor 232 may use these programming instructions in controlling operation of the stimulator. The stimulation subsystem 236 may be configured to generate a stimulation signal and deliver the stimulation signal to a patient via the stimulator probe.

Additionally or alternatively, the control subsystem 136 may comprise a communications subsystem. The communication subsystem may be configured to facilitate communication of data and/or energy between the stimulator and an external source. For example, in some variations the communications subsystem may be configured to allow the stimulator 100 to communicate wirelessly (e.g., via WiFi, Bluetooth, or the like) with an external device (e.g., an external programmer, base station, laptop or other computer, mobile device such as a mobile phone, tablet, wearable computer (e.g., optical head-mounted displays such as Google Glass™) or the like), and may comprise an antenna, coil, or the like. Additionally or alternatively, the communication subsystem may be configured to communicate with an external device (e.g., a flash drive, a laptop or other computer, a mobile device such as a mobile phone, palm pilot, or tablet, or the like) via a wired transmission line. In these variations, the stimulator may comprise one or more ports (e.g., a USB port), connectors and/or cables configured to physically connect the stimulator to an external device, such that data and/or energy may be transmitted between the stimulator and the external device.

As discussed above, some embodiments of the nasal stimulator 100 include a reusable stimulator body 102 and a disposable stimulator probe 104. In such an embodiment, the stimulator probe 104 may releasably couple to the stimulator body 102. For example, the stimulator body 102 may be configured to include the power generator that produces an electrical stimulation waveform, and the stimulator probe may be configured to be inserted in a nasal cavity of a user to deliver the neurostimulation therapy. During use, the disposable stimulator probe 104 may contact nasal mucosa, nasal fluid etc. As such, the stimulator probe 104 may be unsanitary after a single use. Due to the small size and complex geometry of the stimulator probe 104, it may be inefficient and/or not effective for a user to sanitize the stimulator probe 104. As such, routine replacement of the stimulator probe may be required to eliminate risks of biohazard exposure to the user. As such, various safety mechanisms are described herein for authorizing use of stimulator probes, as well as detecting when a stimulator probe needs to be replaced and ensuring replacement of the stimulator probe prior to allowing use of the stimulator.

In some embodiments, the control subsystem 136 may include a safety mechanism (e.g., RFID control mechanism) that limits the duration of use of the stimulator probe 104 to thereby require replacement of the stimulator probe 104, which may limit or prevent biohazard exposure to a user. In some embodiments, the safety mechanisms may be part of the stimulation subsystem 236. The processor 232 may additionally or alternatively comprise software that assists with the performance of the safety mechanism, such as software that tracks and limits use of the stimulator probe 104.

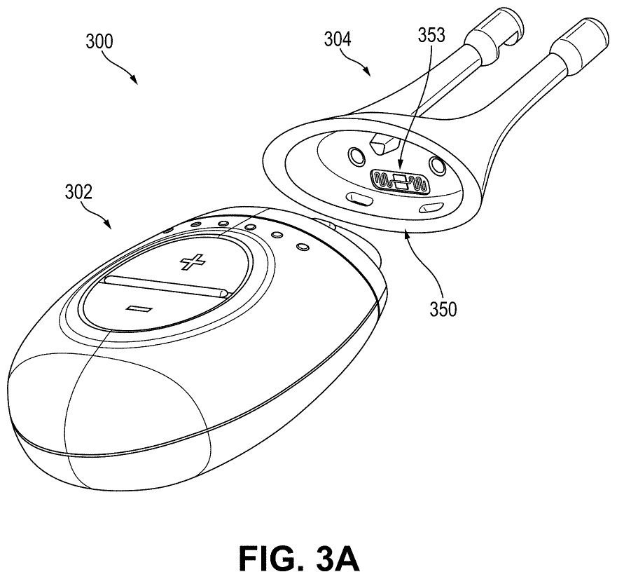

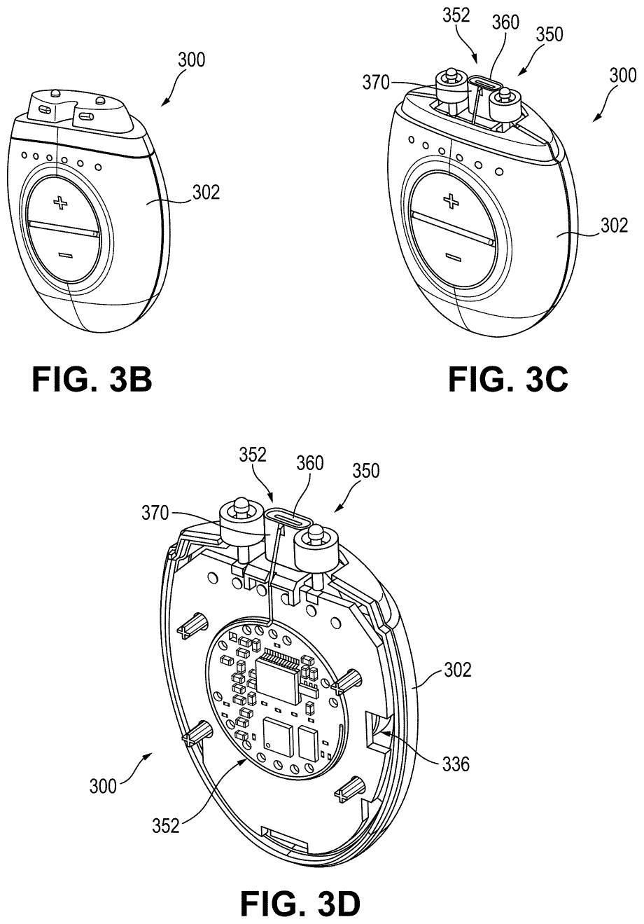

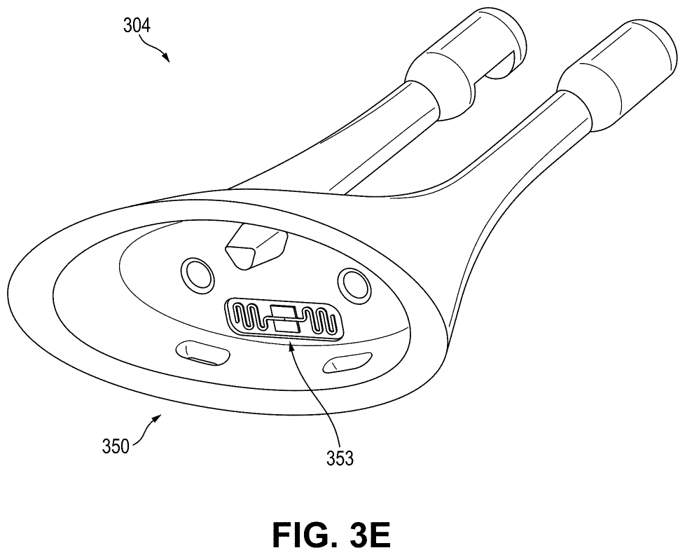

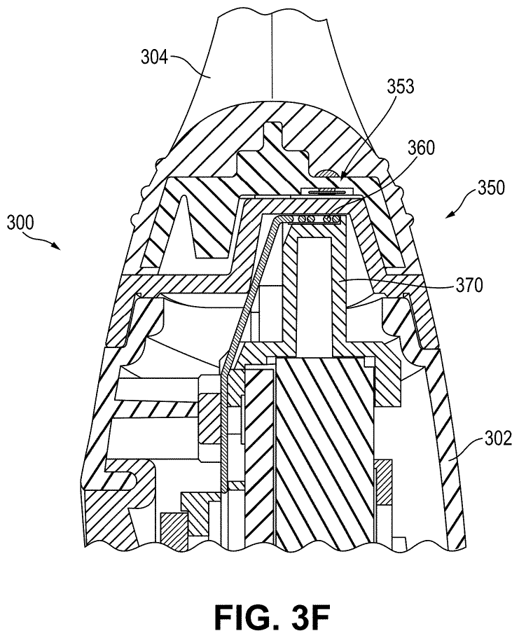

For example, the control subsystem 136 (including the RFID control mechanism) may prevent delivery of current to the stimulator probe when the safety mechanism described with respect to FIGS. 3A-3F detects use of the stimulator probe that exceeds a predefined threshold. For example, the control subsystem 136 may prevent delivery of current (e.g., to the electrodes of the stimulator probe) after detecting the stimulator probe has been coupled to the stimulator body longer than a predefined duration. As such, a user would have to replace the stimulator probe in order for the control subsystem to allow the delivery of current to a stimulator probe. In some variations, the duration may be limited to within a range of approximately one day to approximately thirty days, however, other durations are within the scope of this disclosure. Other safety mechanisms may be included in a stimulator embodiment and may be in communication with the control subsystem 136 for ensuring safe and effective use of the stimulator, as will be described in greater detail below.

In addition to the above contamination concerns, counterfeiting of the stimulator probe 104 is another concern. For example, counterfeit medical devices can be a threat to health and safety due to bypassing safety controls required by the FDA. Other concerns associated with counterfeit medical devices include such devices being provided with improper instructions for use and not being distributed under a licensed practitioner's supervision. As such, the present disclosure describes various safety mechanisms implemented into embodiments of the stimulator that identify and authenticate a stimulator probe before allowing use of the stimulator probe. Such safety mechanisms associated with detecting and limiting the use of a stimulator probe, as well as identifying and authenticating the stimulator probe prior to use is described in greater detail below.

FIGS. 3A-3F illustrate another embodiment of a handheld simulator 300 including a stimulator probe 304 configured to releasably couple to a stimulator body 302. Additionally, the stimulator 300 includes a safety mechanism (e.g., RFID control mechanism) that authenticates and limits the use of the stimulator probe 304 (and any subsequent stimulator probe coupled to the stimulator body). For example, the safety mechanism identifies and authenticates the stimulator probe 304 prior to allowing delivery of stimulation by the stimulator 300, which may prevent against use of counterfeit stimulator probes. Furthermore, after the stimulator probe 304 has been authorized for use, the safety mechanism limits a duration of use of the stimulator probe 304 thereby requiring a user to adhere to replacement requirements of the stimulator probe 304, which may assist with preventing or limiting biohazard exposure to the user.

As shown in FIGS. 3A-3F, the safety mechanism includes a radio-frequency identification (RFID) control mechanism 350 that includes a reader 352 and a tag 353. As shown in FIG. 3A, the stimulator probe 304 may include the tag 353 and, as shown in FIG. 3D, the stimulator body 302 may include the reader 352. The tag 353 may contain electronically stored information, such as a unique tag identifier that is stored on the tag 353 during manufacturing and/or prior to distribution. The tag identifier may be used to identify and authenticate the stimulator probe 304, as will be described in greater detail below.

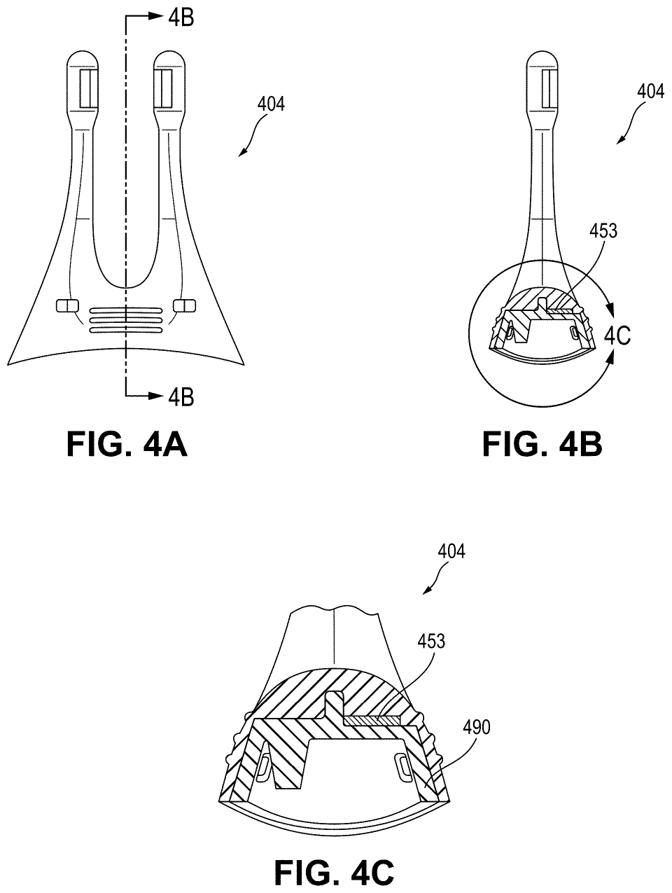

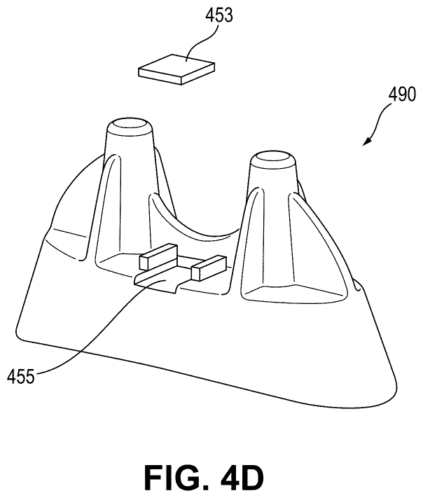

For example, the tag 353 may use radio waves to communicate its identity and other information to the reader 352. The tag 353 may be passive (e.g., does not have a battery or its own power source) and may be powered by the reader 352. The tag 353 may include an integrated circuit (IC) that can store a range of information from one serial number (e.g., tag identifier) to several pages of data. For example, the tag 353 may include an ultra-small Hitachi UHF, Texas instruments Ti HF-1 Type 5 or a Murata HF Magicstrap read-only chip, which may be able to sustain high temperatures and thereby make it suitable for inclusion in a plastic injection molding process used to produce disposable stimulator probes. For example, injection molding a tag into the stimulator probe 304 may effectively embed the tag within the stimulator probe and protect it, such as from corrosive saline solution that may be used in a sterile storage pouch. An example stimulator probe embodiment having a tag embedded therewithin is described in detail below with respect to FIGS. 4A-4D.

As shown in FIGS. 3B-3D, the reader 352 may be included in the stimulator body 302 and may be in communication with the control subsystem 336. The reader 352 may include one or more antennas 360 that emit radio waves and receive signals back from the tag 353. The control subsystem 336 may include a list of acceptable tag identifiers, which may be saved into memory of the control subsystem 336. As such, when the reader 352 communicates with the tag 353 and retrieves the associated tag identifier, the control subsystem 336 may compare the retrieved tag identifier with the stored acceptable tag identifiers to determine if there is a match. In addition the reader 352 itself can write additional information to the tag 353, such as a date the tag was first coupled to the stimulator body 302 and subsequently recognized by the reader 352. If a match is found, the control subsystem 336 may authorize the associated stimulator probe 304 and allow stimulation to be performed (e.g., current may be allowed to be sent to the electrodes of the stimulator probe 304) for the defined service life of the stimulator probe 304. However, if a match is not found or a date code is found that is not valid, the control subsystem 336 may prevent stimulation to be performed using the stimulator probe 304. For example, such failure to match tag identifiers can be due to a counterfeit stimulator probe attempting to be used or the device was previously used. As such, the RFID control mechanism may prevent against use of counterfeit stimulator probes and problems associated with the use of counterfeit stimulator probes.

Furthermore, to ensure a stimulator probe is not re-used, once the stimulator probe is authorized and additional code is written to the tag (e.g., a date stamp), the control subsystem 336 may save the authorized tag identifier in memory. As such, part of an authorization process may include comparing a tag identifier with tag identifiers saved in memory that are identified as having been used and are no longer allowed to be authorized for use. Alternatively or in addition, the control subsystem 336 may remove tag identifiers from a list of acceptable tag identifiers after a match and subsequent authorization has been performed. Other methods and processes for tracking use of authorized stimulator probes to prevent re-use is within the scope of this disclosure.

In some embodiments, the antenna 360 of the reader 352 may be configured to communicate only with a tag 353 associated with a stimulator probe 304 that is coupled to the stimulator body 302. For example, the reader 352 may be configured such that it is unable to communicate with tags associated with stimulator probes that are not coupled to the stimulator body 302. This may assist with ensuring only stimulator probes coupled to the stimulator body are date stamped by the reader 352 for authorization thereby preventing stimulator probes that have not been coupled to the stimulator body from being inadvertently authenticated and identified as being used.

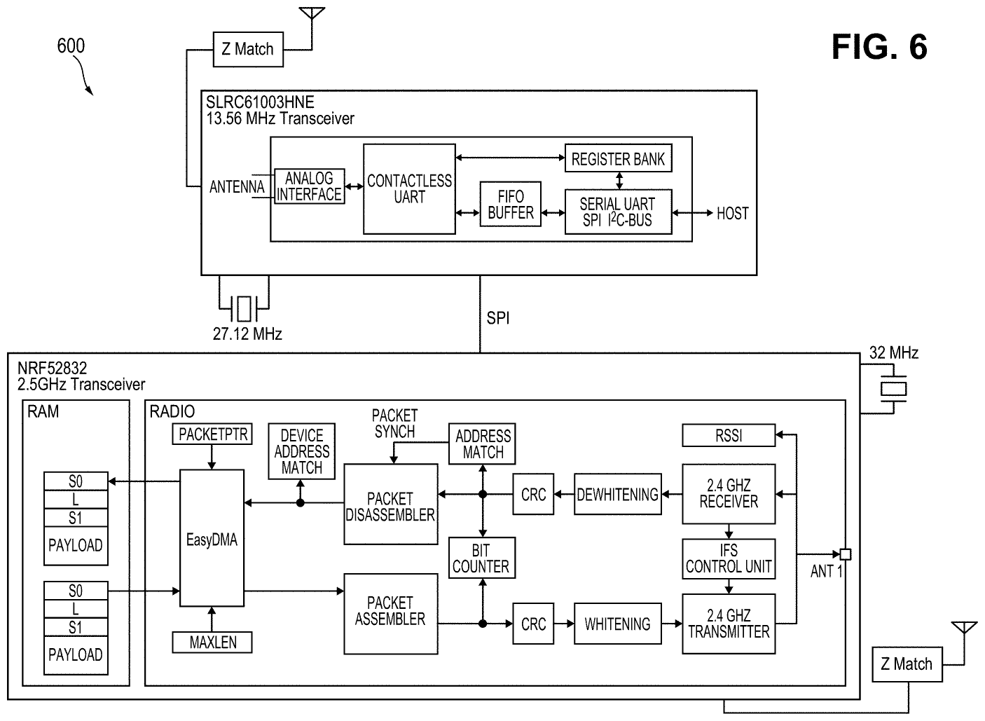

For example, to ensure the tag 353 associated with the stimulator probe 304 coupled to the stimulator body 302 is being identified and authorized, the tag 353 may be placed within a communication range of the reader 352 only when the stimulator probe 304 is coupled to the stimulator body 302, as shown in FIG. 3F. To achieve this configuration, for example, the antenna 360 of the reader 352 may be positioned on a proximal extension 370 adjacent a top, coupling end of the stimulator body 302, as shown in FIGS. 3C and 3D. Additionally, the tag 353 may be positioned along or adjacent a bottom, coupling end of the stimulator probe 304, which may include a cavity for accepting the proximal extension 370, as shown in FIGS. 3E and 3F. As such, when the stimulator probe 304 is coupled to the stimulator body 302, the proximal extension 370 with the antenna 360 can be inserted in the cavity of the stimulator probe 304 for positioning the antennae 360 within the stimulator probe 304, as well as positioning the tag 353 adjacent and within the communication range of the antenna 360. This positioning can allow the reader 352 to communicate with the tag 353 (e.g. retrieve the tag identifier) and initiate authorization of the stimulator probe 304. For example, the communication range may include a distance of 3 mm. In some implementations, the communication range may be less than 3 mm. Other configurations and communication range distances are within the scope of this disclosure, including the communication range achieved by various communication and authorization schematics, such as shown in the block diagram 600 in FIG. 6.

In some embodiments, after the stimulator probe 304 is coupled to the stimulator body 302, date stamped, and authorized for use, the RFID control mechanism may initiate a timer associated with the control subsystem 336. For example, the control subsystem 336 can include software that monitors the timer and allows current to travel to the stimulator probe 304 up until the timer reaches a predefined duration (e.g., 1 hour, 6 hours, 12 hours, 24 hours, 7 days, 30 days, etc.). After such duration is reached, the control subsystem 336 may be configured to prevent current from travelling to the stimulator probe 304. This may force the user to replace the stimulator probe and prevent exposure of biohazard to the user.

In some embodiments, the control subsystem 336 may be configured to track and limit a number of uses of the stimulator probe 304. For example, after the stimulator probe 304 is coupled to the stimulator body 302 and authorized for use, the control subsystem 336 may keep track and count each time the stimulator is activated for providing stimulation. The control subsystem 336 may be configured to prevent stimulation after a predefined number of uses is reached (e.g., one use, five uses, ten uses, etc.). Other variations of the control system for tracking and limiting use of the stimulator probe are within the scope of this disclosure.

An example method of use of the stimulator 300 may include a first step of attaching the stimulator probe 304 to the stimulator body 302. Thereafter, the stimulator body 302 may be powered on, which may initiate the RFID control mechanism, such as activate the reader 352 to search for and communicate with the tag 352 associated with the stimulator probe 304. If communication with the tag 352 is established and a tag identifier is retrieved, the control subsystem 336 may authenticate and validate the tag identifier, such as by comparing the tag identifier with a list of acceptable tag identifiers stored in memory. If the tag is validated as authentic and previously unused, code may be written to the tag to thereby allow use of the stimulator probe 304. In addition, the control subsystem 336 may activate a time clock associated with a programmed duration time-limit or threshold, as discussed above. Stimulation may then be delivered by the stimulator until the duration threshold is reached. For example, once the duration threshold is reached, the control subsystem 336 may activate a feedback element of the stimulator (e.g., illuminate one or more LED) to notify the user that a new stimulator probe is required. After the duration threshold is reached, the control subsystem 336 may prevent stimulation delivery until a new stimulator probe is attached to the stimulator body and authorized for use.

In some embodiments, the stimulator body may be programmed to identify authentic disposable stimulator probes. Such programming may be achieved by downloading tag identifiers that are authorized for use onto the reader and/or control subsystem. For example, such downloading of tag identifiers can be achieved via Bluetooth using a tablet or smart phone to sync with a complimentary Bluetooth enabled stimulation base. In some embodiments, the stimulator body may include a re-writeable RFID reader component and an additional longer range antenna. A separate RFID reader may be configured to communicate with the re-writeable RFID reader component. For example, this separate RFID reader may be a stand-alone device located at a clinic, a pharmacy, and/or added to a tablet or smart phone.

In some embodiments, the stimulator body can include a processor and communication subsystem of the control subsystem 336 that allows data to be downloaded and stored on the stimulator body, such as one or more lists of authorized tag identifiers. For example, when a user is prescribed a stimulator and provided with a unit of stimulator probes, such as from a prescription or provided by a licensed doctor, the control subsystem 336 of the stimulator body may be updated with a list of tag identifiers associated with the unit of stimulator probes provided to the user. The stimulator body may be updated in a variety of ways, including any variation described above.

FIGS. 4A-4D illustrate another embodiment of a stimulator probe including a tag 453 of an RFID control mechanism 450, both of which include any of the features and/or functions described herein with regards to the RFID control mechanism 350 and tag 353 of FIGS. 3A-3F (which will not be repeated here for sake of brevity). As discussed above, to ensure the tag 453 associated with the stimulator probe 404 coupled to a stimulator body (such as the stimulator body 302 of FIG. 3A) is being identified and authorized, the tag 453 may be placed within a communication range of the reader only when the stimulator probe 404 is coupled to the stimulator body. To achieve this configuration, the tag 453 may be embedded within the stimulator probe 404, as shown in FIGS. 4B and 4C. As such, when the stimulator probe 404 is coupled to the stimulator body, the tag 453 is positioned adjacent and within the communication range of the antenna of the reader thereby allowing the reader to communicate with the tag 453 (e.g. retrieve the tag identifier) and initiate authorization of the stimulator probe 404. For example, the communication range may include a distance of 3 mm. In some implementations, the communication range may be less than 3 mm. Other communication range distances are within the scope of this disclosure.

For example, during manufacturing the tag 453 can be coupled to a first part of the stimulator probe 404 and thereafter overmolded with a material to thereby embed the tag 453 within the stimulator probe, as shown in FIG. 4C. As shown in FIGS. 4C and 4D, the stimulator probe can include a platform 490 made out of a first material and have one or more features for positioning and/or coupling the tag 453 thereto. As shown in FIG. 4D, the platform 490 can include a recessed feature 455 that is sized and shaped to allow the tag 453 to couple thereto. Such recessed feature 455 can assist with properly securing a position of the tag 453 during manufacturing, such as to ensure the tag 453 is embedded within the stimulator probe 404 at a position that allows for the reader to communicate with the tag 453 when the stimulator probe 404 is coupled to the stimulator body, as discussed above.

Various embodiments of the coupling or recessed feature 455 can be included in the platform 490. For example, the recessed feature 455 can be sized and shaped to provide a snap or friction fit between the tag 453 and at least two extruded sides surrounding or forming a part of the recessed feature 455. In some embodiments, the recessed feature 455 can provide a surface for the tag 453 to be adhered thereto. For example, once the tag 453 is coupled to the recessed feature 455, a molding process can be performed to embed the tag 453 into the stimulator probe 404. For example, the tag 453 coupled to the recessed feature 455 can be overmolded with a material, such as a material used to form the simulation prongs. The platform and overmold material can include any number of materials, including any number of plastic and/or medical grade materials.

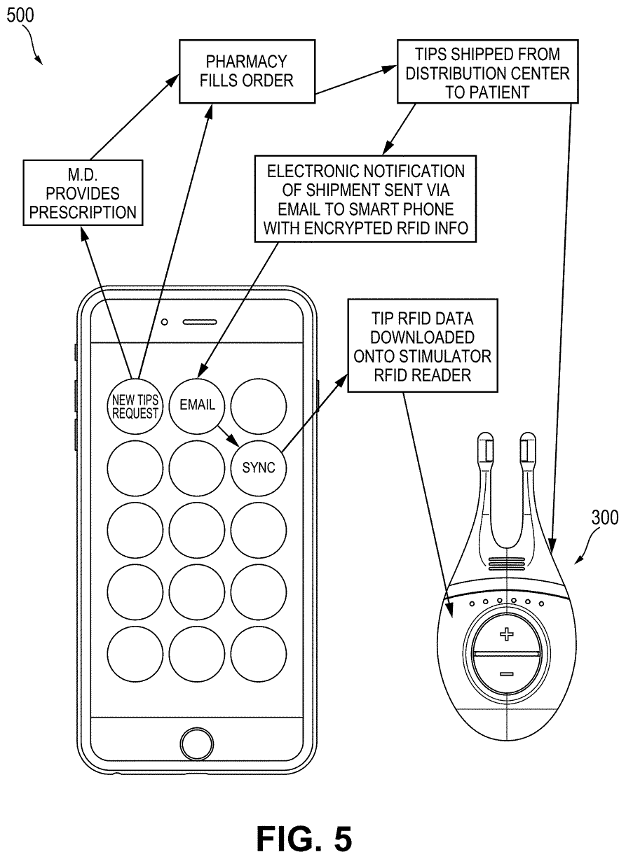

FIG. 5 shows an information flow diagram 500 illustrating data that may be sent from a smartphone to the stimulator body of the handheld stimulator 300. For example, as shown in FIG. 5, the list of unique tag identifiers can be downloaded from an electronic device (e.g., a smartphone) onto the stimulator body 302 of the handheld stimulator 300 for enabling the stimulator body 302 to authorize use of acceptable stimulator probes 304 (e.g., a stimulator probe including a tag having a tag identifier that matches one of the unique tag identifiers in the list of unique tag identifiers downloaded by the electronic device). For example, a medical service provider can provide the information containing the list of unique tag identifiers to the electronic device for allowing the download of the list of unique tag identifiers, such as in conjunction with a prescription provided to a patient associated with the smartphone. Various other information can be provided to and from the stimulator body and is within the scope of this disclosure.

FIG. 6 illustrates a block diagram 600 schematically representing a variation of the handheld stimulator, including the communication and authorization schematics associated with the RFID control mechanisms of the stimulator probe and stimulator body. The communication and authorization schematic can include radios associated with the stimulator probe and/or stimulator body. As discussed above, the RFID control mechanism can assist with controlling use of stimulator probes, such as preventing, allowing, and/or limiting use of stimulator probes with the stimulator body. Other variations of the RFID control mechanism, including various communication and authorization schematics, are within the scope of this disclosure.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A handheld stimulator system for stimulating nasal tissue of a subject, comprising:
a stimulator probe, wherein the stimulator probe comprises:
a first nasal insertion prong comprising a first electrode; and
a tag of an RFID control mechanism, the RFID control mechanism configured to control a use of the stimulator probe; and
a stimulator body configured to releasably couple the stimulator probe thereto, wherein the stimulator body comprises:
a power source; and
a reader of the RFID control mechanism, the reader being configured to communicate with the tag for assisting with controlling the use of the stimulator probe.

2. The handheld stimulator system of claim 1, wherein the tag includes a tag identifier that is read by the reader for authenticating the stimulator probe.

3. The handheld stimulator system of claim 2, wherein the tag is embedded within a material comprising the stimulator probe.

4. The handheld stimulator system of claim 2, wherein the tag is positioned adjacent a base of the stimulator probe, wherein the base couples with a coupling end of the stimulator body.

5. The handheld stimulator system of claim 4, wherein the reader is positioned along the coupling end of the stimulator body thereby allowing the reader to communicate with the tag when the stimulator probe is coupled to the stimulator body.

6. The handheld stimulator system of claim 1, wherein the reader is configured to communicate with the tag when the tag is positioned within a predefined distance of the reader.

7. The handheld stimulator system of claim 6, wherein the tag is positioned at the predefined distance when the stimulator probe is coupled to the stimulator body, and wherein the tag is outside of the predefined distance when the stimulator probe is separated from the stimulator body.

8. The handheld stimulator system of claim 4, wherein an antennae of the reader is positioned adjacent an end of an extension extending from the coupling end of the stimulator body, wherein the extension is configured to be inserted within a cavity along the base of the stimulator probe for positioning the antennae within the stimulator probe and adjacent the tag.

9. The handheld stimulator system of claim 1, wherein the RFID control mechanism is configured to compare a received identifier associated with the tag against a list of stored acceptable tag identifiers and allow a current from the power source to be delivered to the first electrode if the received identifier matches an acceptable tag identifier of the list of stored acceptable tag identifiers.

10. The handheld stimulator system of claim 1, wherein the RFID control mechanism includes a programmed predefined threshold that controls a delivery of power from the power source to the first electrode.

11. The handheld stimulator system of claim 10, wherein the RFID control mechanism is configured to prevent current from the power source to the first electrode after a use parameter of the stimulator probe meets and/or exceeds the predefined threshold.

12. The handheld stimulator system of claim 11, wherein the predefined threshold defines a duration of time or a date.

13. The handheld stimulator system of claim 11, wherein the predefined threshold defines a number of times a stimulation is delivered.

14. The handheld stimulator system of claim 1, wherein the RFID control mechanism is configured to prevent current from the power source to the first electrode if an identifier associated with the tag fails to match an acceptable tag identifier in a list of acceptable tag identifiers stored on the stimulator body.

15. The handheld stimulator system of claim 1, wherein the RFID control mechanism is configured to include a use identifier on the tag after the stimulator probe is coupled to the stimulator body for assisting with limiting the use of the stimulator probe.

16. The handheld stimulator system of claim 1, wherein the stimulator probe includes a second nasal insertion prong comprising a second electrode.

17. A method of a handheld nasal stimulator, comprising:
coupling a stimulator probe of the handheld nasal stimulator to a stimulator body of the handheld nasal stimulator, the stimulator probe comprising an electrode and a tag associated with an RFID control mechanism, the RFID control mechanism configured to control a use of the stimulator probe;
detecting, after the coupling and by a reader of the RFID control mechanism, the tag of the stimulator probe, the stimulator body including the reader configured to communicate with the tag when the tag is positioned within a predefined range from the reader;
comparing, by the RFID control mechanism, an identifier associated with the tag against a list of acceptable identifiers stored on the stimulator probe; and
controlling, by the RFID control mechanism and based on the comparing, a delivery of current from a power source of the stimulator body to the electrode of the stimulator probe for delivering a stimulation.

18. The method of claim 17, wherein the controlling includes allowing the delivery of current from the power source to the electrode when the identifier associated with the tag matches an acceptable identifier of the list of acceptable identifiers.

19. The method of claim 18, wherein the controlling further includes preventing the delivery of the current from the power source to the electrode when a use parameter of the stimulator probe meets and/or exceeds a programmed predefined threshold.

20. The method of claim 19, wherein the programmed predefined threshold is a number of times the stimulation is delivered.

21. The method of claim 19, wherein the programmed predefined threshold is a duration of time or a date.

22. The method of claim 17, wherein the controlling includes preventing the delivery of current from the power source to the electrode when the identifier associated with the tag fails to match an acceptable identifier of the list of acceptable identifiers.

23. The method of claim 17, further comprising associating, by the reader and after the coupling, a use identifier with the tag for limiting the use of the stimulator probe.
